(19) United States
(12) Patent Application Publication
(10) Pub. No.: US 2009/0018346 A1
(43) Pub. Date: Jan. 15, 2009

(54) Amorphous Form Of Darifenacin Hydrobromide And Processes For The Preparation Thereof

(75) Inventors: Monica Benito Velez, Barcelona (ES); Stephen Benedict David Winter, Barcelona (ES); Nuria Soldevilla Madrid, Barcelona (ES)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Medichem, S.A., Sant Joan Despi (ES)

(21) Appl. No.: 12/171,366

(22) Filed: Jul. 11, 2008

Related U.S. Application Data
(60) Provisional application No. 60/959,379, filed on Jul. 13, 2007.

(51) Int. Cl. C07D 405/10

(52) U.S. Cl. 548/525

(57) ABSTRACT

The present invention relates to amorphous form darifenacin hydrobromide and processes therefor. In addition, the present invention relates to compositions comprising amorphous form darifenacin hydrobromide.

### CROSS-REFERENCE TO RELATED APPLICATIONS

This patent application claims the benefit of U.S. Provisional Patent Application No. 60/959,379, filed Jul. 13, 2007, which is incorporated herein by reference.

### BACKGROUND OF THE INVENTION

Darifenacin is an active pharmaceutical substance indicated for the treatment of overactive bladder with symptoms of urge urinary incontinence, urgency and frequency. Darifenacin is the international common accepted name for (S)-2-{1-[2-(2,3-dihydrobenzofuran-5-yl)ethyl]-3-pyrrolidinyl}-2,2-diphenylacetamide. The hydrobromide salt of darifenacin has been selected for medical purpose. Darifenacin hydrobromide has an empirical formula of C₂₈H₃₀N₂O₂—HBr, and the structure represented below.

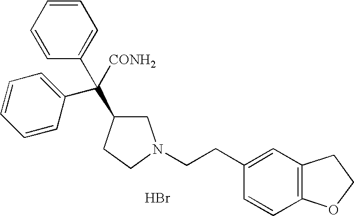

In the United States, darifenacin hydrobromide is marketed under the trade name of ENABLEX® and in Europe as EMSELEX®.

Darifenacin and its pharmaceutically acceptable salts are reported in U.S. Pat. No. 5,096,890 (“the '890 patent”). The '890 patent discloses a preparation of darifenacin hydrobromide wherein darifenacin hydrobromide is isolated after treatment of a solution of darifenacin in acetone with 49% aqueous hydrobromic acid. However, the '890 patent reports only the melting point of the obtained solid darifenacin hydrobromide (229° C.), but does not disclose the polymorphic form.

U.S. Pat. No. 6,930,188 (“the '188 patent”) reports a stable hydrate of darifenacin, as well as a toluene solvate of darifenacin. In the '188 patent, darifenacin hydrobromide is prepared by addition of 48% aqueous hydrobromic acid to a solution of darifenacin hydrate or darifenacin toluene solvate in butan-2-one. However, the '188 patent reports only the melting point of the solid obtained as 232° C. and the IR data, but does not disclose the polymorphic form.

The publication IPCOM 000137408D (Jun. 19, 2006) (“the '408D publication”) reports the DSC, X-Ray Powder Diffraction, FT-IR, particle shape and particle size data of crystalline darifenacin hydrobromide. The '408D publication reports that the crystalline solid is precipitated from acetone. The list of the main XRD peaks of darifenacin hydrobromide precipitated from methanol, acetonitrile or dichloromethane is also reported. Hereinafter this form will be designated as Form I.

According to IPCOM 000137408D crystalline darifenacin hydrobromide Form I is characterized by powder X-ray diffraction peaks: 8.2, 9.1, 11.5, 12.5, 14.4, 16.7, 17.1, 17.3, 17.8, 18.3, 18.8, 19.2, 19.6, 20.1, 20.3, 20.8, 22.1, 23.7, 24.1, 24.7, 25.2, 25.9, 26.2, 26.8, 27.4, 27.6, 28.1, 28.9, 30.0, 30.3±0.2 degrees 2θ.

The '890 and '188 patents, as well as the '408D publication are incorporated herein by reference.

Polymorphism is defined as the ability of a substance to crystallize in more than one crystal lattice arrangement. Polymorphism can influence many aspects of solid state properties of a drug. Different crystal modifications of a substance may differ considerably from one another in many respects such as, for example, solubility, dissolution rate, and bioavailability. An exhaustive treatment of polymorphism in pharmaceutical and molecular crystals is given for example by Byrn (Byrn, S. R., Pfeiffer, R. R., Stowell, J. G., “Solid-state Chemistry of Drugs” 2^{nd} edition, SSCI Inc., West Lafayette, Ind., 1999), by Brittain, H. G., “Polymorphism in Pharmaceutical Solids”, Marcel Dekker, Inc., New York, Basel, 1999) or by Bernstein (Bernstein, J., “Polymorphism in Molecular Crystals”, Oxford University Press, 2002), the disclosures of which are incorporated herein by reference.

Crystalline solids normally require a significant amount of energy for dissolution due to their highly organized lattice like structures. For example, the energy required for a drug molecule to escape from a crystal is much higher than the energy required for escaping from an amorphous form.

Additionally, it is known that amorphous forms of a number of drugs exhibit different solubility properties, and in some cases also exhibit different bioavailability patterns, as compared to their crystalline form. For some therapeutic indications, one bioavailability pattern may be favored with respect to another. Therefore, it is desirable to have amorphous forms of drugs and processes for their preparation.

Accordingly, there is a need to provide amorphous form darifenacin hydrobromide, as well as processes for preparing amorphous form darifenacin hydrobromide, and compositions comprising amorphous form darifenacin hydrobromide.

### BRIEF SUMMARY OF THE INVENTION

The present invention provides amorphous darifenacin hydrobromide, processes therefor, and compositions comprising amorphous darifenacin hydrobromide.

In some embodiments, the present invention provides amorphous form darifenacin, including pharmaceutically acceptable salts thereof, such as, for example, amorphous form darifenacin hydrobromide. In other embodiments, the present invention provides amorphous darifenacin hydrobromide having an X-ray diffraction pattern and IR spectrum as described herein.

Other embodiments of the invention provide a process for preparing amorphous darifenacin hydrobromide which comprises melting crystalline darifenacin hydrobromide and cooling the molten darifenacin hydrobromide.

In other embodiments, the present invention provides a process for preparing amorphous darifenacin hydrobromide comprising removing solvent from a solution of darifenacin hydrobromide.

In other embodiments, the present invention provides a process for preparing darifenacin hydrobromide crystalline Form I from amorphous darifenacin hydrobromide.

In other embodiments, the present invention provides pharmaceutical compositions comprising amorphous darifenacin hydrobromide.

### BRIEF DESCRIPTION OF THE DRAWINGS

FIG. 1 illustrates the X-ray powder diffraction (XRD) of darifenacin hydrobromide Form I.

FIG. 2 illustrates the IR spectrum of darifenacin hydrobromide Form I.

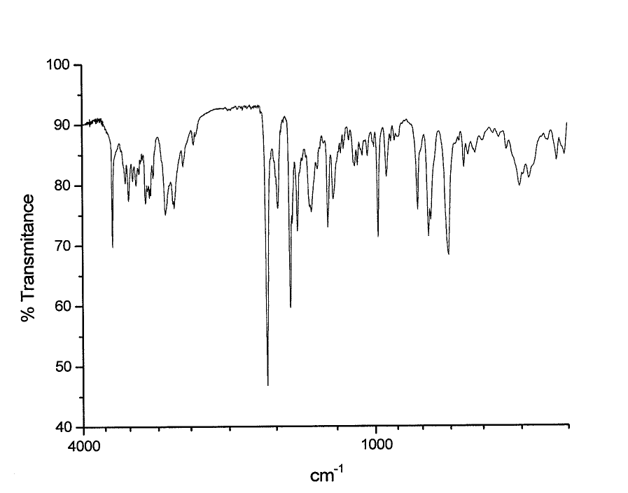

FIG. 3 illustrates the X-ray powder diffraction (XRD) of amorphous form darifenacin hydrobromide.

FIG. 4 illustrates the IR spectrum of amorphous form darifenacin hydrobromide.

### DETAILED DESCRIPTION OF THE INVENTION

As used herein, the phrases “amorphous darifenacin hydrobromide” and “amorphous form darifenacin hydrobromide” have the same meaning and are used interchangeably.

In accordance with an aspect of the invention, the present invention provides amorphous darifenacin hydrobromide.

In some embodiments, amorphous darifenacin hydrobromide is characterized by an X-ray diffraction pattern.

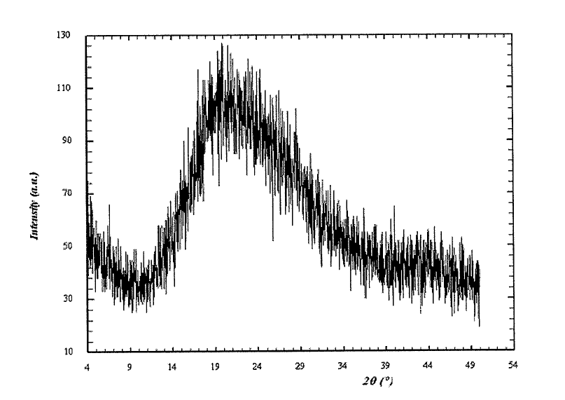

As described herein, typically amorphous darifenacin hydrobromide is characterized by a broad X-ray diffraction maximum at about 10 to about 35 degrees 2θ, as shown in FIG. 3. In preferred embodiments, amorphous darifenacin hydrobromide has an X-ray diffraction pattern (2θ) as substantially shown in FIG. 3.

In some embodiments, amorphous darifenacin hydrobromide is characterized by an IR spectrum.

In accordance with some embodiments, amorphous darifenacin hydrobromide is characterized by an IR spectrum having peaks (cm⁻¹) at approximately: 3472, 3292, 3223, 3057, 3018, 2936, 2856, 2671, 2569, 2471, 1672, 1589, 1493, 1445, 1360, 1242, 1219, 1105, 1036, 982, 943, 901, 818, 754, 704, 662, 640, 569, 530, 498, 419.

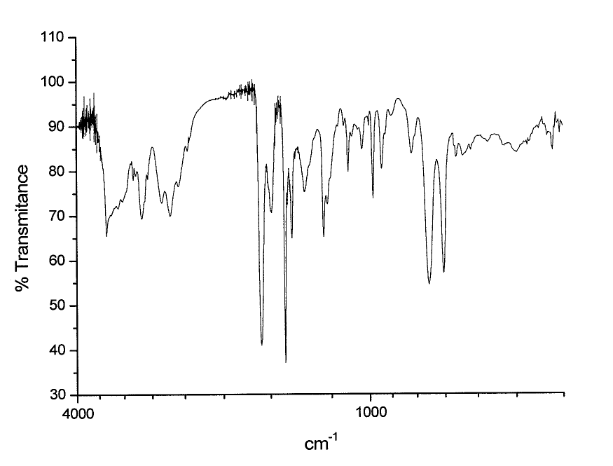

In preferred embodiments, amorphous darifenacin hydrobromide has an IR spectrum as substantially shown in FIG. 4.

In some embodiments, amorphous darifenacin hydrobromide is characterized by both an XRD and IR spectrum.

In accordance with other aspects of the invention, the present invention provides processes for preparing amorphous darifenacin hydrobromide. In some embodiments, amorphous darifenacin hydrobromide is prepared using darifenacin hydrobromide which is obtained from a known method. In other embodiments, amorphous darifenacin hydrobromide is prepared using darifenacin hydrobromide crystalline Form I.

In some embodiments, amorphous form darifenacin hydrobromide is obtained by heating darifenacin hydrobromide to a temperature at which the darifenacin hydrobromide melts and cooling the molten darifenacin hydrobromide. The temperature at which darifenacin hydrobromide is heated in order to melt the darifenacin hydrobromide is about 235° C.

Typically, the molten darifenacin hydrobromide is cooled by removing the heating source and allowing the material to cool to below room temperature. In some embodiments, molten darifenacin hydrobromide is cooled using an ice-bath. In a preferred embodiment, molten darifenacin hydrobromide is cooled to a temperature between about room temperature and about 0° C. In other preferred embodiments, the molten darifenacin hydrobromide is cooled to a temperature of about 0° C.

As known in the art, room temperature refers to a temperature range of about 20° C. to about 25° C.

Upon cooling, the solidified amorphous material can be isolated, ground or milled, and/or sieved if desired.

In a preferred embodiment, the invention provides a process for preparing amorphous darifenacin hydrobromide comprising melting darifenacin hydrobromide by heating darifenacin hydrobromide to a temperature wherein the darifenacin hydrobromide melts and cooling the molten darifenacin hydrobromide to a temperature between about room temperature and about 0° C., thereby forming amorphous darifenacin hydrobromide.

In other embodiments the invention provides a process for preparing amorphous darifenacin hydrobromide which comprises providing darifenacin hydrobromide, forming a solution of darifenacin hydrobromide in a suitable solvent, and removing the solvent from the solution.

In some embodiments, the solution of darifenacin hydrobromide is prepared by dissolving darifenacin hydrobromide in a suitable solvent. For example, in some embodiments crystalline darifenacin hydrobromide (e.g., crystalline Form I) is dissolved in a suitable solvent and then the solvent is removed from the solution, thereby forming amorphous darifenacin hydrobromide.

In other embodiments, darifenacin hydrobromide is provided by preparing a solution of darifenacin hydrobromide from darifenacin free base or other pharmaceutically acceptable salts of darifenacin. In these embodiments, darifenacin free base or other pharmaceutically acceptable salts thereof are dissolved in a suitable solvent and then converted in situ to darifenacin hydrobromide. The solvent is then removed from the solution of darifenacin hydrobromide, thereby forming amorphous darifenacin hydrobromide.

Typically, the solvent is an organic solvent. In preferred embodiments the solvent is selected from the group consisting of methanol, chloroform, dichloromethane, and mixtures thereof.

The removal of the solvent can be effected by simple evaporation or by more intensive procedures. In an embodiment, the solvent is removed by evaporating the solvent. In a preferred embodiment, the solvent is removed by heating the solution under vacuum.

In other embodiments, the solvent is removed by spray drying the solution of darifenacin hydrobromide. In spray drying a solution of darifenacin hydrobromide, the solvent is preferably a C₁-C₅ alcoholic solvent or mixtures thereof, more preferably the solvent is methanol. The spray drying equipment can operate at different conditions such as, for example, with an inlet temperature from 50° C. to 150° C., and a feed rate from 1 to 50 mL/min.

In keeping with another aspect of the invention, the present invention also provides a process for preparing darifenacin hydrobromide crystalline Form I. In some embodiments, darifenacin hydrobromide crystalline Form I is prepared from amorphous darifenacin hydrobromide. In accordance with these embodiments, amorphous darifenacin hydrobromide is exposed to conditions of relative humidity, temperature, and a period of time sufficient to convert amorphous darifenacin hydrobromide to darifenacin hydrobromide crystalline Form I.

In a preferred embodiment, the relative humidity is about 75%, the temperature is about 40° C., and the period of time is about 14 days.

It will be appreciated by those skilled in the art that the various conditions of relative humidity, temperature, and period of time can be varied so long as darifenacin hydrobromide crystalline Form I is formed.

The present invention provides pharmaceutical compositions. In preferred embodiments, the present invention provides a pharmaceutical composition comprising amorphous darifenacin hydrobromide.

The following examples further illustrate the invention but, of course, should not be construed as in any way limiting its scope.

The XRD diffractograms were obtained using a RX SIEMENS D5000 diffractometer with a vertical goniometer, a copper anodic tube, and radiation CuK_{α}, λ=1.54056 Å.

Fourier transform IR spectra were acquired on a Shimadzu FTIR-8300 spectrometer, and polymorphs were characterized in potassium bromide discs.

### EXAMPLE 1

This example illustrates a process for preparing amorphous darifenacin hydrobromide in accordance with an embodiment of the invention.

Darifenacin hydrobromide crystalline Form I was placed in a vial, which was heated using an oil bath until the darifenacin hydrobromide melted. The vial containing the molten darifenacin hydrobromide was cooled for 30 minutes in an ice-bath resulting in the formation of a solid. After cooling, the solid was ground to give amorphous darifenacin hydrobromide.

These results demonstrate that amorphous darifenacin hydrobromide can be prepared from darifenacin hydrobromide crystalline Form I.

### EXAMPLE 2

This example illustrates a process for preparing amorphous darifenacin hydrobromide in accordance with an embodiment of the invention.

Solutions of crystalline darifenacin hydrobromide were prepared according to Table 1. Crystalline darifenacin hydrobromide was dissolved in the solvent identified in Table 1 at the concentration indicated therein.

TABLE 1

| | | Concentration | XRPD |
|---|---|---|---|
| Sample | Solvent | (mg/mL) | result |
| 1 | chloroform | 50.1 | Amorphous |
| 2 | dichloromethane | 35.8 | Amorphous |
| 3 | methanol | 34.5 | Amorphous |
| 4 | chloroform | 200 | Amorphous |

The solvent from each sample was removed from the solution by heating (40° C.) under vacuum. The obtained solid from each sample was analyzed by means of X-ray powder diffraction (XRPD). The diffractogram is shown in FIG. 3.

These results demonstrate that amorphous darifenacin hydrobromide can be prepared from a solution of darifenacin hydrobromide.

### EXAMPLE 3

This example illustrates a process for preparing darifenacin hydrobromide Form I from amorphous darifenacin hydrobromide in accordance with an embodiment of the invention.

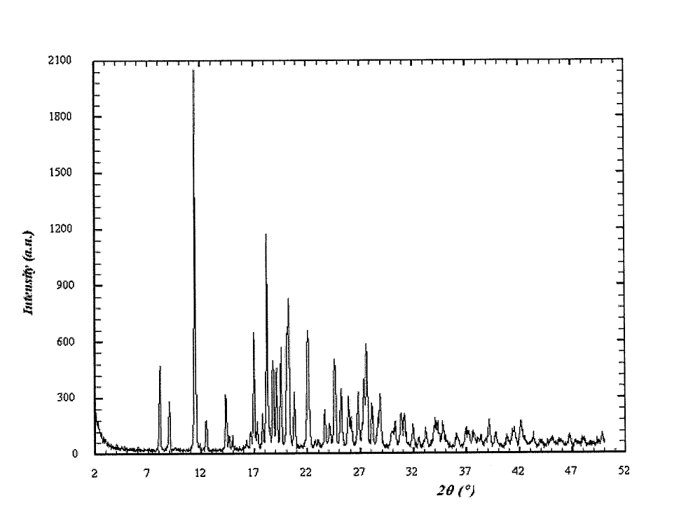

A sample of amorphous darifenacin was placed in an open vial in a desiccator within an incubator at a temperature of 40° C. The desiccator contained saturated aqueous salt solutions to maintain the relative humidity to about 75%. After 14 days the sample was removed and analyzed by XRPD. The diffractogram is shown in FIG. 1.

This result demonstrates that darifenacin hydrobromide Form I can be prepared from amorphous darifenacin hydrobromide.

### EXAMPLE 4

This example illustrates a process for preparing amorphous darifenacin hydrobromide in accordance with an embodiment of the invention.

A solution of darifenacin hydrobromide (4.8 g) in methanol (150 mL) was spray dried using a Buchi B290 spray dryer equipped with Buchi inert loop B295 operating in closed loop mode with nitrogen gas.

The following parameters were used: inlet temperature (actual) of 60° C.; outlet temperature (actual) of 42° C.; aspirator of 100% (equivalent to approximately 35 m³/hour); nozzle cleaning of 5; nitrogen flow of 30 mm (equivalent to approximately 440 L/hour); and inert loop condenser temperature (set point) of −20° C. The peristaltic pump to feed the product solution was set to 15% (equivalent to approximately 5 mL/min).

The obtained solid was analyzed by means of X-ray powder diffraction (XRPD). The diffractogram was in accordance with the diffractogram shown in FIG. 3.

These results demonstrate that amorphous darifenacin hydrobromide can be prepared from a solution of darifenacin hydrobromide.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Amorphous form darifenacin hydrobromide.

2. Amorphous form darifenacin hydrobromide having an X-ray diffraction pattern (2θ) as substantially shown in FIG. 3.

3. Amorphous form darifenacin hydrobromide having an X-ray diffraction pattern (2θ) having a characteristic broad X-ray diffraction maximum at about 10 to 35 degrees 2θ.

4. A process for preparing amorphous darifenacin hydrobromide comprising:
i) melting crystalline darifenacin hydrobromide, and
ii) cooling the molten darifenacin hydrobromide to a temperature between about room temperature and about 0° C., thereby forming amorphous darifenacin hydrobromide.

5. A process for preparing amorphous darifenacin hydrobromide comprising:
i) providing darifenacin hydrobromide, and
ii) dissolving darifenacin hydrobromide in a solvent, and
iii) removing the solvent from said solution, thereby forming amorphous darifenacin hydrobromide.

6. The process of claim 5, wherein the solvent is selected from the group consisting of methanol, chloroform, dichloromethane, and mixtures thereof.

7. The process of claim 5, wherein the solvent is removed by evaporation.

8. The process of claim 5, wherein the solvent is removed by spray drying.

9. The process of claim 4, wherein the darifenacin hydrobromide used for preparing amorphous darifenacin is crystalline darifenacin hydrobromide.

10. The process of claim 5, wherein the darifenacin hydrobromide used for preparing amorphous darifenacin is crystalline darifenacin hydrobromide.

11. The process of claim 9, wherein the crystalline darifenacin hydrobromide is darifenacin hydrobromide crystalline Form I.

12. The process of claim 10, wherein the crystalline darifenacin hydrobromide is darifenacin hydrobromide crystalline Form I.

13. A process for preparing darifenacin hydrobromide crystalline Form I comprising exposing amorphous darifenacin hydrobromide at a relative humidity, temperature, and a period of time sufficient to convert amorphous darifenacin hydrobromide to darifenacin hydrobromide crystalline Form I.

14. The process of claim 13, wherein the relative humidity is about 75%.

15. The process of claim 13, wherein the temperature is about 40° C.

16. The process of claim 13, wherein the period of time is about 14 days.

17. A pharmaceutical composition comprising amorphous darifenacin hydrobromide.

18. The process of claim 5, wherein the darifenacin hydrobromide is provided by an in situ conversion of darifenacin free base or other pharmaceutically acceptable salt of darifenacin to darifenacin hydrobromide.
